(19) United States
(12) Patent Application Publication
(10) Pub. No.: US 2011/0112557 A1
(43) Pub. Date: May 12, 2011

(54) APPARATUS AND METHOD FOR LAPAROSCOPIC PORT SITE SUTURE

(76) Inventor: Antony Hugh Beeley, Siesta Park (AU)

(21) Appl. No.: 13/001,312

(22) PCT Filed: Jul. 7, 2009
(86) PCT No.: PCT/AU09/00859

§ 371 Date: Dec. 23, 2010

(30) Foreign Application Priority Data
Jul. 2, 2008 (AU) 2008903408

(51) Int. Cl. A61B 17/04; A61M 29/00

(52) U.S. Cl. 606/148; 606/191; 606/228

(57) ABSTRACT

A surgical device for guiding a needle during suturing of an incision comprising a body for insertion in the incision having a first passage to conduct an end of the needle below a location surrounding the incision, wherein a distal portion of the body comprises means for indicating the location to which the needle will be conducted. Methods for suturing an incision using the surgical device are also provided.

### FIELD OF THE INVENTION

The present invention relates to the medical field and more particularly to apparatus and methods applied during surgical procedures for suturing incisions

### BACKGROUND ART

Invasive surgical procedures which involves the cutting of one or more incisions to access a biological cavity are widespread. The size of different types of incisions may vary depending on the type of surgical procedure being performed. For example, it might be desirable or required to perform only small incisions in order to access a biological cavity. A patient's body may be punctured in order to insert, for example, a telescope into the patient's body, to view the inside of a biological cavity. Also, a small incision may be made to insert in the biological cavity a tube that delivers gas (usually CO₂) in order to insufflate the cavity thus providing a viewing space for the telescope and a working space for surgical instruments provided via other small incisions.

As an example, laparoscopic surgery (key hole surgery) procedures are performed by passing instruments down hollow tubes inserted through small incisions made in an abdominal wall. These tubes are called ports. The ports may vary in diameter from 0.5 cm to 1.5 cm. As previously explained, the abdomen is insufflated through the small incisions in order to provide a working and viewing space within the abdomen.

Upon completion of the laparoscopic surgical procedure, the incision(s) may need to be sutured. The suture method is cumbersome and difficult because the incisions are usually small and deep. Thus, a surgeon typically is unable to get his fingers into the incision to perform the suturing procedure. Instead, a surgeon must pass a needle and suture through the tissue on one side of the incision and into the patient's body; manipulate the needle and suture with forceps via the small incision, and then pass the needle and suture through the tissue on the other side of the incision and out of the body.

Moreover, when ports having diameters of 1 cm or more, especially in the lower part of the abdomen (below the level of the umbilicus), there is a high risk of developing a hernia through the port site (port site hernia). Also, it is occasionally necessary to enlarge a port site by stretching it with a dilator in order to deliver for example a large gallstone (3 or 4 cm in diameter). In such cases a port site hernia is almost certain to occur unless the muscle and peritoneum of the port site is properly sutured. Further, a port site hernia will require a further operation at a later date to repair it and this may be a very difficult procedure.

Port site hernias can be avoided by suturing the muscle layer together with the peritoneum (lining of the inner aspect of the abdominal wall) of the port site. In order to suture them properly it is important that the working and viewing space created by insulffation of the cavity be maintained through continual pumping of the gas into the cavity (as the gas gradually escapes during the procedure, primarly through the incisions). Enlargement of the incisions will result in an increased discharge of gas from the abdomen and therefore in a reduction of the working and viewing space. Also, when performing the suturing of the incisions the ports must be extracted from the incisions, allowing exit of the gas and thus reducing even more the working and viewing space. This creates a series of inconveniences when suturing the patient. Upon release of the gas, the abdominal wall lies in contact with the intra abdominal organs such as the bowel so that the risk of picking up the bowel wall during the suture procedure of the incisions is greatly increased.

### SUMMARY OF THE INVENTION

According to a first aspect of the invention there is provided a surgical device for guiding a needle during suturing of an incision comprising a body for insertion in the incision having a first passage to conduct an end of the needle below a location surrounding the incision, wherein a distal portion of the body comprises means for indicating the location to which the needle will be conducted.

Preferably, the indicating means may comprise a marking on the distal portion.

Preferably, the location is adjacent to the marking.

Preferably, the first passage traverses obliquely the body.

Preferably, the first passage traverses the body at an angle of about 30° with respect to a side wall of the body.

The indicating means may comprise a first mark for providing a visual indication of a specific depth of the body within the incision.

The indicating means may comprise a second mark for providing a visual indication of an angular rotation of the body thereby providing an indication of the location to which the end of the needle will be conducted.

There may be a series of first marks in spaced apart relation.

There may be two opposed second marks.

Preferably, each of the opposed second marks extend from the first passage to a distal end of the body.

Preferably, the distal end is configured for entry into the incision.

Preferably, the distal end is of tapered configuration.

Preferably, the body is adapted to sealingly engage the incision.

In one arrangement the body further comprises a second passage.

Preferably, the second passage traverses obliquely the body at a second angle relative to the longitudinal axis of the body,

Preferably, the second angle being different than a first angle at which the first passage traverses obliquely the body.

In a further arrangement, the body comprises three or more passages, each of the three or more passages traversing obliquely the body at angles relatively to the longitudinal axis of the body.

Preferably, the angles being different from each other.

Preferably, the passages are arranged to conduct the end of the needle to a same location relative to the incision.

Preferably, the same location is located 1.5 cm radially outwards from the the marking

Preferably, the body comprises transparent material.

The body may be defined by a trocar.

According to a second aspect of the invention there is provided a surgical device for guiding a needle during suturing of an incision comprising a body for insertion in the incision having a plurality of passages to conduct an end of the needle below a location surrounding the incision, wherein a distal portion of the body comprises means for indicating the location to which the needle will be conducted.

Preferably, the plurality of passage traverses obliquely the body at angles relatively to the longitudinal axis of the body,

Preferably, the angles being different with respect to each other.

According to a third aspect of the invention there is provided a dilator for determining the diameter of an incision, the dilator comprising at least one mark indicating the diameter of the dilator at the location of the at least one mark.

According to a fourth aspect of the invention there is provided a tool for guiding a surgical instrument with respect a biological cavity, the tool comprising at least one end adapted to receive the surgical instrument.

According to a fifth aspect of the invention there is provided a method for suturing an incision in a biological cavity comprising the steps of:
obturating the incision to avoid gas discharge from the biological cavity; and
suturing the incision.

According to a sixth aspect of the invention there is provided a method for suturing an incision in a biological cavity comprising the steps of:
inserting a body in the incision having at least one first passage to conduct a first end of a thread to a location below the incision; the body being inserted into the incision until at least one first mark on the body reaches a location of the incision;
inserting a first end of a thread inside the biological cavity through the at least one first passage;
extracting a second end of the thread from the at least one passage;
rotating the body around its longitudinal axis until at least one second mark on the body reaches a location around the incision;
extracting the first end of the thread outside of the biological cavity through the at least one first passage;
extracting the body from the incision;
uniting the first end of the thread with a second end of the thread for closing of the incision.

Preferably, the previous steps are repeated a plurality of times for finishing to complete closure of the incision using at least one thread.

Preferably, the first end of the thread is inserted into the biological cavity attached to a distal end of a needle.

Preferably, the distal end of the needle comprises fastening means to releasably attach a thread.

Preferably, fastening means to releasably attach a thread comprise at least one indentation having at least one extension extending into the body of the needle onto which the thread is mounted.

Preferably, the first end of the thread is removed from the distal end of the needle.

Preferably, the distal end of the needle is extracted from the at least one first passage.

Alternatively, the distal end of the needle is extracted from incision but kept within the passage of the body so as to allow rotation of the body.

Preferably, the body is raised to extract a second end of the thread from the at least one passage.

Preferably, the body is rotated 180 degrees around its longitudinal axis.

Preferably, the first end of the thread is extracted from the biological cavity through the at least one first passage via the distal end of the needle.

Preferably, the method further comprises inserting a telescope into the biological cavity for inspection inside the cavity.

Alternatively, the telescope is inserted inside the body for inspection inside the biological cavity.

Preferably, the method further comprises inserting a forceps into the biological cavity for either removing the thread from the distal end of the needle or hooking the thread to the needle.

Preferably, the first end of the thread is released from the distal end of the needle via the forceps.

Preferably, the first end of the thread is mounted on the distal end of the needle via the forceps.

Preferably, the method further comprises delivering gas into the biological cavity.

Preferably, the method is performed during laparoscopic surgery.

Preferably, the method is performed on a port site incision.

Preferably, the suturing comprises suturing the muscle layer and the peritoneum of the abdomen.

Preferably, the method further comprises the step of selecting the diameter of the body by use of a dilator.

Preferably, the dilator is a graded dilator.

Preferably, the body is defined by a trocar.

According to a seventh aspect of the invention there is provided a method for suturing an incision in a biological cavity comprising the steps of
inserting a body in the incision having at least one first passage to conduct a first end of a thread to a location below the incision;
inserting a first end of a thread inside the biological cavity through the at least one first passage;
extracting a second end of the thread from the at least one passage;
rotating the body around its longitudinal axis until at least one second mark on the body reaches a location around the incision;
extracting the first end of the thread outside of the biological cavity through the at least one first passage;
extracting the body from the incision;
uniting the first end of the thread with a second end of the thread for closing of the incision.

According to an eight aspect of the invention there is provided a method for guiding at least one portion of a surgical instrument into a biological cavity, the method comprising:
inserting a guide having an end adapted to receive at least one end of the surgical instrument;
contacting the end of the guide against a location of a wall of the biological cavity;
making an incision at the location;
inserting the at least one end of the surgical instrument in the incision;
contacting the end of the guide against the at least one end of the surgical instrument; and
inserting the at least one portion of the surgical instrument in the cavity through the incision with assistance of the guide.

### BRIEF DESCRIPTION OF THE DRAWINGS

The present invention will be better understood by reference to the following description of several specific embodiments thereof as shown in the accompanying drawings, in which:

FIG. 1 is a schematic view of a fragmentary cross-section of the body of a patient at a location surrounding an incision to be sutured, with various apparatus and devices used in a surgical procedure being conducted on the patient shown schematically in position;

FIG. 2 is a view of an apparatus according to a first embodiment of the invention inserted into the incision;

FIG. 3 is front view of the apparatus according to the first embodiment of the invention;

FIG. 4 is back view of the apparatus according to the first embodiment of the invention;

FIG. 5 is a view of an apparatus according to a second embodiment of the invention inserted into the incision;

FIG. 6 is front view of the apparatus according to the second embodiment of the invention;

FIG. 7 is back view of the apparatus according to a second embodiment of the invention;

FIG. 8 is a detail of a cross-section of an arrangement of the surgical instruments according to the first or second embodiment;

FIG. 9 is a side view of a dilator for use in conjunction with the apparatus according to the first or second embodiment of the invention;

FIG. 10 is a side of a tool for locating an incision and guiding a surgical instrument;

FIG. 11 is an end view of a tool for locating an incision and guiding a surgical instrument according to a fifth embodiment of the invention;

FIGS. 12 to 23 are successive diagrammatic sectional views illustrating the method for suturing of an incision with single sutures using the surgical instrument according to the first embodiment of the invention;

FIG. 24 is a top view of an incision being sutured by a method using the first embodiment of the invention;

FIG. 25 is a top view of an incision sutured by a method using the first embodiment of the invention;

FIGS. 26 to 51 are successive diagrammatic sectional views illustrating the method for suturing of an incision with a continuous suture using the surgical instrument according to the first embodiment of the invention; and

FIGS. 52 to 54 are successive diagrammatic sectional views illustrating the method for providing a surgical instrument to a body according to an eighth embodiment of the invention.

### DETAILED DESCRIPTION OF SPECIFIC EMBODIMENT(S)

The embodiments to be described relate to apparatus and methods for suturing incisions.

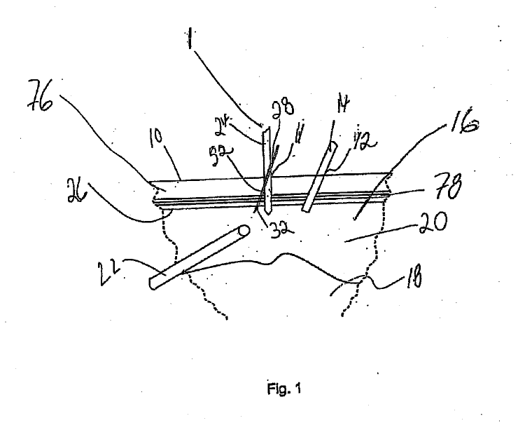

FIG. 1 is a schematic cross-sectional view of a section of the abdomen of a patient undergoing laparoscopic surgery. In the surgical procedure, a first incision 11 is made in the abdominal wall 10 of the patient, which subsequently needs to be closed. Apparatus 1 according to the first embodiment of the invention is used in the procedure for closing the incision 11. As part of the laparoscopic surgical procedure second and third incisions perforate the abdominal wall for insertion of a port 14 and telescope 22. The port 14 facilitates delivery of instruments such as forceps (not shown) into the abdomen 16 for the procedure. Organs 18 within the abdomen are separated from the abdominal wall 10 by a space 20. Space 20 provides the working space required to perform the suture procedure and the viewing space so that the telescope 22 may provide images of the procedure within the abdomen 16. It should be noted that FIG. 1 is schematic only and the location and disposition of the apparatus 1, port 14 and telescope 22 can vary according to the location and type of surgical procedure being performed.

A gas may be delivered into the abdomen to maintain the space 20, thus not allowing contact between the abdominal wall 10 and the organs 18.

The incision 11 is sutured with the aid of the apparatus 1. The apparatus 1 is inserted into the incision 11, as will be explained.

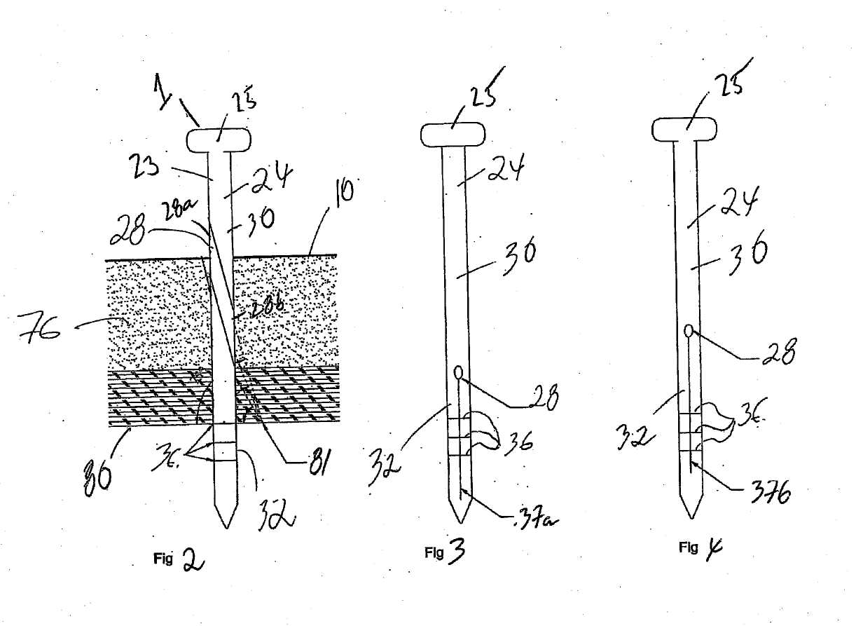

Referring to FIGS. 2 to 3, the apparatus 1 according to a first embodiment comprises a body 24. The body 24 is located at a specific height with respect, for example, the inner surface 26 of the abdominal wall.

The body 24 comprises an elongated portion configured as a shaft 30 of circular cross-section. The shaft 30 has a distal end 32 which defines a conical section 34.

The body 24 comprises a passage 28 adapted to receive a needle 52 and guide a distal end of the needle 52 below a location surrounding the incision. The passage 28 has opposed ends 28a, 28b opening onto opposed sides of the body 24. Passage 28 traverses the body 24 obliquely.

The distal end 32 of the body 24 comprises markings 36, 37. The markings comprise one or more first marks 36 configured as horizontal marks. In the arrangement shown there are three horizontal marks 36a, 36b and 36c spaced apart along the distal end of the needle 52. The marks 36 may be seen with the aid of the telescope 22 when the body 24 is inserted in the incision 11.

The marks 36 provide an indication of the depth of insertion of the elongated portion 30 in the incision 11 and thus facilitate accurate suture placement. The marks 36 allow a surgeon to adjust the depth at which the distal end of the needle 52 will perforate the peritoneum 80. The adjustment in depth of body 24 allows accurate suturing of any depth of incision. Varying the depth of insertion of the body within the incision controls the location at which the distal end of the needle 52 perforates the side walls of the incision 11. This allows adjustment of the location of the body 24 to the thickness of the muscle layer that a particular patient has.

One or more second marks 37 are provided on the body 24 to provide a visual indication of the angular orientation of the body and thereby provide an indication of the azimuthal location of the needle 52. In the arrangement shown, there are two vertical marks 37a and 37b disposed in diametrically opposed relation; that is, the vertical marks 37a and 37b are located in opposed sides of the body 24 (see FIGS. 3, 6 and 4, 7). This provide a surgeon with an indication as to the location at which the distal end of the needle 52 will emerge from the tissue surrounding the incision 11 and enter the abdominal cavity.

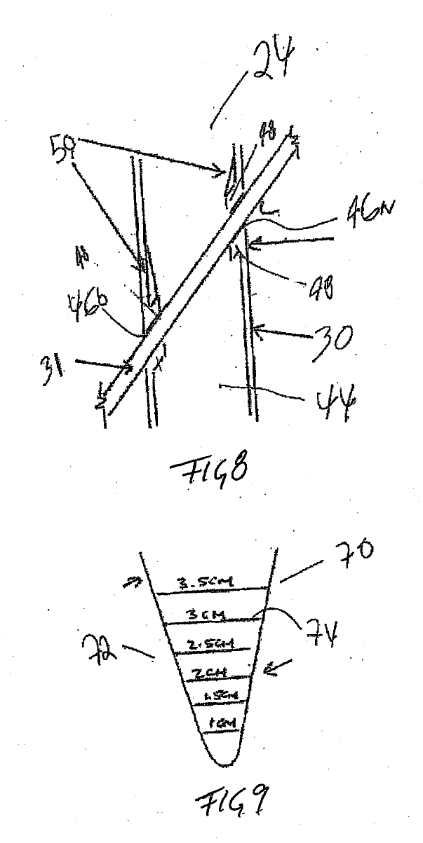

The first vertical mark 37a extends from the end 28a of passage 28 to the distal portion 32 of the body 24 (see FIG. 6). The first vertical mark 37a is visible by the telescope and provides en visual indication of indicates the location at which the distal end of the needle 52 will pierce the peritoneum 80 (for example, 1.5 cm radially outwards form the first vertical mark 36b). The second vertical mark 37b extends from the opposing end 28b of passage 28 to the distal portion of the body 24 (see FIG. 4).

The first vertical mark 37a and the second vertical mark 37b have different lengths. This allows the first and second vertical marks 37a and 37b to be visually distinguished from each other. This is useful while suturing the incision 11. As will be explained with reference to the method for suturing the incision 11, after inserting the distal end of the needle 52 at a first suture site for delivery of a tread 38 (see FIG. 12) the body 24 is rotated to form a second suture site. The second vertical mark 37b provides an indication up to which degree the body 24 has to be rotated in order that the second suture site will be formed opposite to the first suture site.

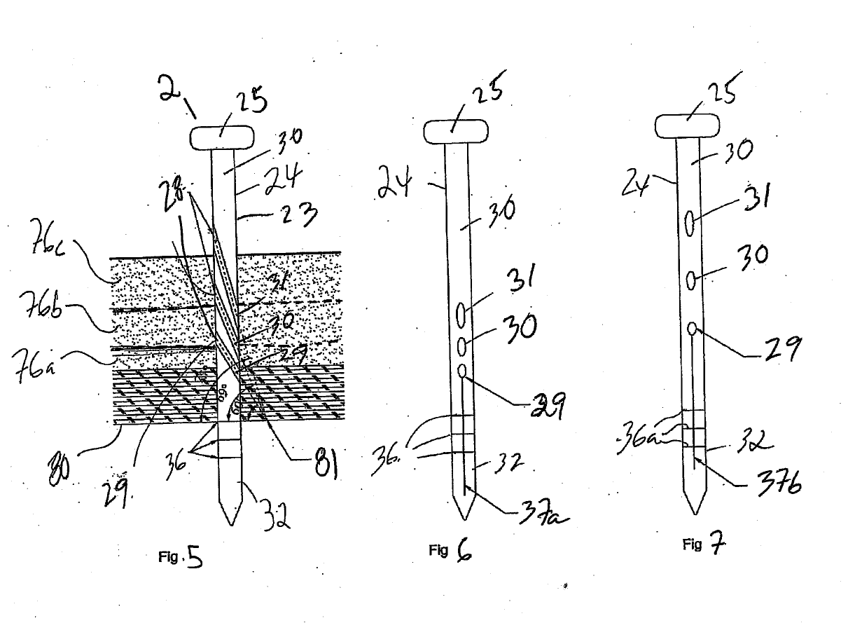

In the arrangement shown in FIGS. 2 to 4 passage 28 traverses the elongate element 30 at an angle of about 30° with respect to the longitudinal axis of the elongate element 30. In other arrangements the passages 28 may traverse the elongate element 30 at angles other than 30°. Differently oriented passages 28 take into account the different level of obesity in patients. Obese patients may require relatively steeper passages 28, as will be explained with reference to FIG. 5.

The body 24 is of uniform diameter along its length to permit axial movement of the body 24 within the incision 11 during insertion and any adjustment of the depth of penetration, as well as rotation within the incision. This may allow maintenance of sealing contact between the body 24 and the body tissue surrounding the incision to avoid loss of air from the pneumoperitoneum.

Moreover, the protruding proximal portion 23 of the body 24 extends above the abdominal wall 10 allowing the body 24 to be held byhe surgeon's hand. The surgeon, to achieve a greater bite of muscle and peritoneum, may push the body 24 sideways while maintaining the elongate element 30 generally perpendicular to the abdominal wall 10. Upper portion 25 of the body 24 may be configured to assist in the movement of the body 24 while the surgeon locates the body 24 within the incision 11 and subsequently performs the suture process.

Also, the protruding proximal portion 23 of the body 24 allows verification that the body 24 is retained generally perpendicular to the abdominal wall 10. Tilting of the body 24 is to be avoided, otherwise the distal end of the needle 52 might either exit the peritoneum at a non-suitable location or, if the tilt is excessive, the needle will stay in the abdominal wall and fail to enter the peritoneal cavity.

FIGS. 5 to 7 show an apparatus 2 according to a second embodiment of the invention. In the arrangement shown in FIGS. 5 to 7, the apparatus 2 comprises a body 24 is similar to the body 24 previously described and shown in FIGS. 2 to 4, and so similar reference numerals are used to identify similar parts.

The body 24 of apparatus 2 according to the second embodiment of the invention comprises a plurality of passages 28 traversing the body 24. In the arrangement shown there are three passages 28 comprising first passage 29, second passage 30 and third passage 31. The passages 29, 30 and 31 are oriented at different angles with respect to each other. As shown in FIG. 5, passages 30 and 31 traverse the body 24 at steeper angles than passage 29. However, the passages 29, 30 and 31 are arranged such that the distal end of the needle 52 (traversing any of the passages 28, 29 and 31) arrives adjacent the body 24 at a same location 81 on the peritoneum 80, as depicted in FIG. 5. For example, a suitable location is positioned about 1.5 cm radially from the intersection of the vertical mark 37 with the horizontal mark 36a.

The plurality of passages 28 allow the body 24 to be used with a range of patients, regardless of the thickness of the patient's fat layer. For example, FIG. 5 shows an abdominal wall 10 with three different fat layers (76a, 76b and 76c). Fat layer 76a might be of a slim patient, fat layer 76b of a normal patient and fat layer 76c of an obese patients. To suture an incision 11 in the slim patient passage 28 may be used. In the normal patient passage 28 will be covered by the fat layer 76b, thus, passage 29 may be used for suturing a patient. In an obese patient the only available passage is passage 30 because the fat layer 76c covers passage 28 and 29.

The body 24 according to the first and second embodiment of the invention may be a biocompatible material such as an appropriate plastics material or metal such as stainless steel.

An alternative arrangement of the first and second embodiment of the invention is shown in FIG. 8. FIG. 8 shows a body 24 similar to the body 24 previously described and shown in FIG. 2 and similar reference numerals are used to identify similar parts. Referring to FIG. 8, the body 24 comprises a hollow tubular element 30 having counterpart apertures 46a and 46b adapted to define the passage 28 for receiving a needle 52. The apertures 46a and 46b are located each at opposite locations of the body 24 and at different heights with respect to each other such that the needle 52 transverses obliquely the body 24. Apertures 46a and 46b include at the inside of the body 24 guiding and sealing means to guide the distal end of the needle 52 trough the body 24 and to avoid gas discharge through the apertures 46. As shown in FIG. 8, the guiding and sealing means comprise cuffs 48 that provide a tight seal around the needle 52. According to this arrangement, a telescope and/or other surgical instruments (not shown) may extend through the tubular body 24 to the distal end (not shown) of the body 24. For this the distal end (not shown) thereof (when, of course, the passage 28 is not occupied by the needle 52) may comprise a transparent section that permits viewing the area below the incision. Guiding means for guiding the telescope through the body 24 are provided inside the body 24. The guiding means are bevels 50 located adjacent to cuffs 48 to direct the distal end of the telescope (not shown) away from cuffs 48 as the telescope is delivered through the body 24.

In another arrangement, the gas may be delivered to the abdomen trough the tubular body 24. For this the distal end (not shown) of the body 24 comprises an aperture (not shown) for delivery of the gas to the abdomen. Also, the proximal end (not shown) of the body 24 may comprise a valve system to prevent gas from escaping the abdomen via the body 24 and permitting the introduction of, for example, surgical instruments and/or a telescope.

A third embodiment of the invention (which is not shown) a trocar is configured to incorporate the passage(s) 28 and also the marks 36 and 37. The trocar may be supplied with a port. With such an arrangement, the passage(s) and the marks 36 are located at the distal end of the trocar. This enables the trocar, supplied with the port, to be used for suturing its incisions (instead of a separate body 24). Trocars are regularly used to introduce ports in abdominal walls. Thus, with this embodiment it is possible to suture the incision in which the ports are inserted with the same trocar used to insert the port. In this way it is not necessary to have a separate surgical instrument, such as body 24, to suture the port site. Passage(s) 28 and marks 36 and 27 may be included in any type of trocar. Alternatively, the trocar may include a single passage and/or any of marks 36 or 37.

In FIG. 9 there is shown a dilator 70 used for increasing the size of an incision for extraction of, for example, a large specimens such as excised colons or gallstones. The dilator 70 may also be used to establish the diameter of the body 24, trocar or any other surgical instrument required for suturing an incision after the extraction of the specimen. The dilator 70 comprises a distal end 72 conical in shape and having a plurality of marks 74 that extend at spaced intervals along the longitudinal axis of the dilator 70. Associated with each mark each mark 74 is an indication of the diameter of the dilator at the location of the mark 74. This provides and indication of the diameter of an incision after it has been increased via the dilator. In operation, the user inserts the dilator 70 into the incision until the desired expansion of the incision is reached. A telescope can provide an image of the area below the incision showing the mark 74 of the dilator 70 that coincides, for example, with the lower surface of the abdominal wall (not shown) thus providing an indication of the expanded diameter of the incision 11. This permits the user to choose a body 24, trocar or any other surgical instrument that has the diameter measured by the dilator and which is suitable for insertion in incision 11.

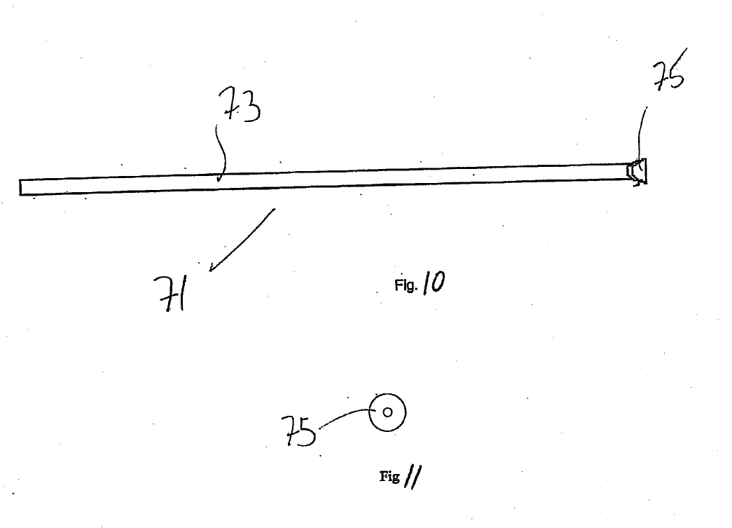

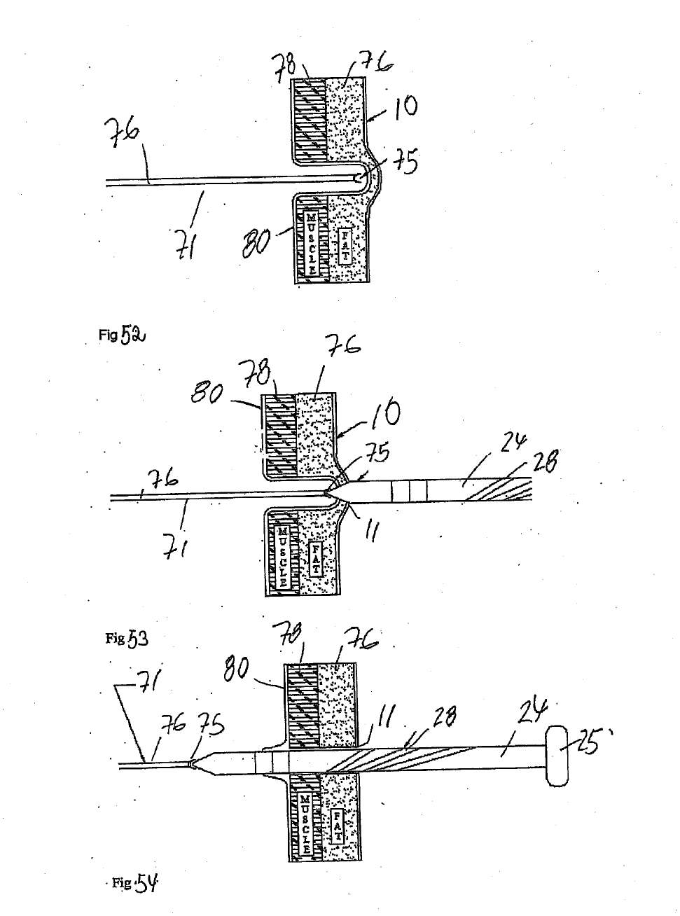

FIGS. 10 and 11 show a tool 71 adapted to indicate the location of an incision to be made for insertion of a surgical instrument. The tool 71 is also adapted to guide the surgical instrument 24 though the incision 11 (see FIGS. 52 to 54).

The tool 71 comprises a rod 73 having an end 75 adapted to receive a surgical instrument. In the arrangement shown, the end 75 of the tool is cup shaped. The surgical instrument may be, for example, a body 24, a trocar as previously described, among others.

FIGS. 12 to 23 illustrate the method for suturing of an incision using an apparatus 1 according to the first embodiment of the invention. As shown in the FIGS. 12 to 23 an abdominal wall 10 comprises a fat layer 76, a muscle layer 78 and the peritoneum 80 (lining of the inner aspect of the abdominal wall). As previously described, in order to avoid complications and further surgery it is important that the muscle layer 78 be sutured with the peritoneum 80, being careful to ensure that no organ 18 is caught during the suture procedure. Thus, during suturing of the incision, the gas content in the abdomen must be kept substantially constant so the working and viewing space 20 is maintained. This may be accomplished by obturating the incision to avoid gas discharging from the abdomen during the suturing process.

As shown in FIG. 12, the body 24 is introduced in the incision 11. The abdominal space 20 is maintained and the site to be sutured may be viewed from within by the telescope 22 which have been inserted in the abdomen via another incision (see FIG. 1). Also, the risk for puncturing or sewing up an internal organ is greatly reduced.

Referring to FIG. 12, the body 24 is inserted into the incision 11 at a specific height with respect to, for example, the inner surface of the abdominal wall. The marks 36 on the body 24 can be sighted through the telescope 22, providing a visual indication of the extent of insertion of the body 24 into the incision 11. THe extent of insertion of the body 24 varies according to the thickness of abdominal musculature of the patient. In this particular arrangement the first horizontal mark 36a coincides with the inner lining (peritoneum) 80 of the abdominal wall 10. This arrangement is, for example, for an abdominal wall of a patient of average abdominal musculature. If the patient has a thicker than average musculature the body 24 must be positioned within the incision 11 at a different depth, such as that indicated by another one of the marks 36.

Vertical marks 37 (see for example FIGS. 6 and 7), also sighted through the telescope, indicate the location at which the distal end of the needle 52 perforates the tissue surrounding the body 24. Alternatively, the apparatus 1 according to the second embodiment of the invention or a trocar according to the third embodiment of the invention may be used.

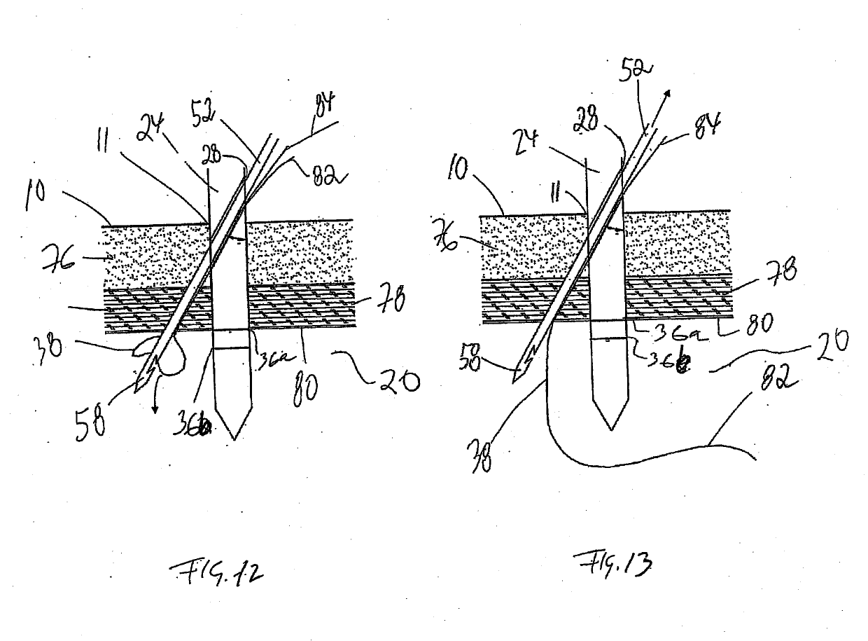

Having adjusted the depth of the body 24 inside the incision 11, a first end 82 of a thread 38 is inserted into the abdomen 16 via the passage 28 of the body 24 (see FIGS. 12 and 13). This is accomplished by the following steps: releasably attaching the thread 38 to the distal end 58 of the needle 52; inserting the distal end 58 of the needle 52 with the thread 38 into the passage 28 of the body 24; and locating the distal end 58 of the needle 52 inside the abdomen 16, as shown in FIG. 12. In the abdomen, the thread 38 is released from the distal end 58 of the needle 52. This may be accomplished via a forceps (not shown) which have been introduced inside the abdomen via a port 14 (see FIG. 1). Also, the first end 82 of the thread 38 may be pulled with the forceps out of the passage 28 of the body 24 and located inside the abdomen 16 (see FIG. 13).

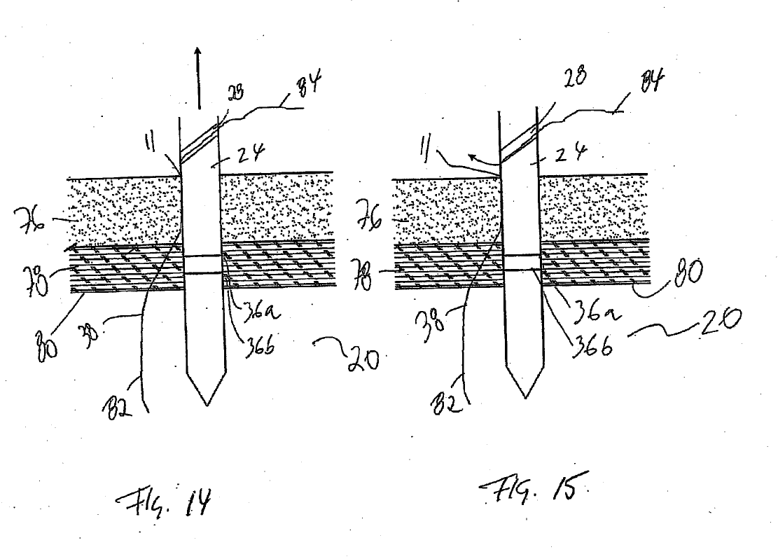

Referring to FIGS. 14 and 15, once the first end 82 of the thread 38 is located inside the abdomen 16, the distal end 58 of the needle 52 is extracted from the abdomen 16 and the passage 28. As shown in FIG. 14, the body 24 is raised permitting to extract the second end 84 of the thread 38 out of the passage 28 of the body 24.

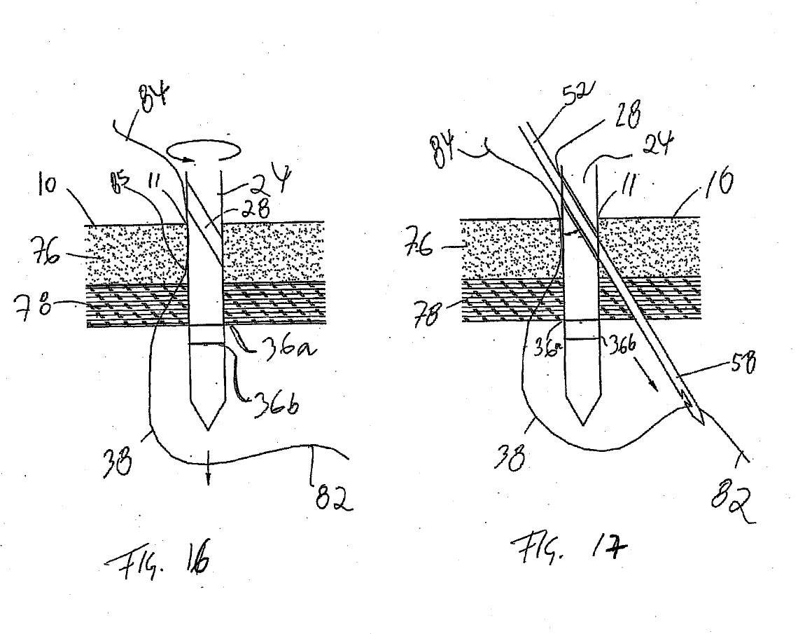

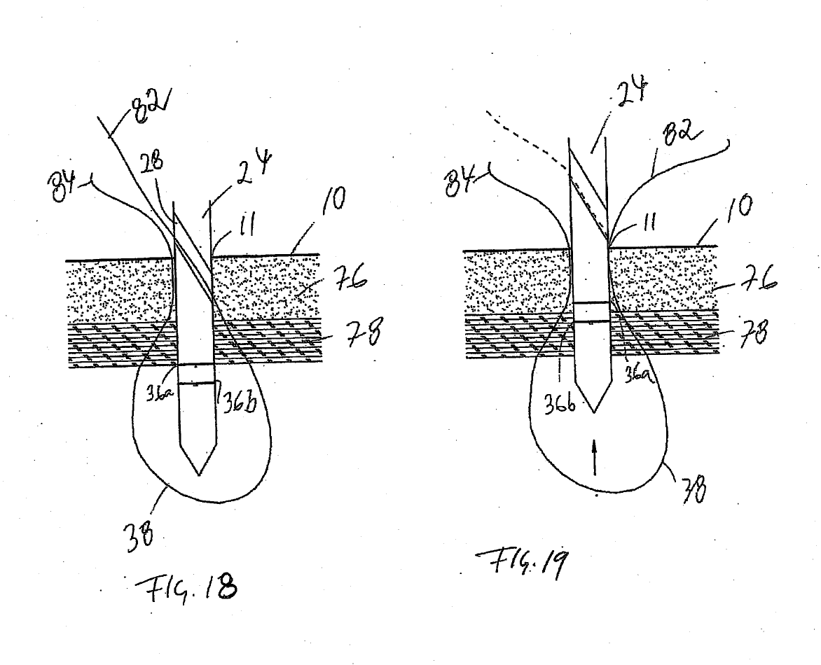

As shown in FIG. 16, the body 24 is rotated through 180°. As previously explained, the vertical marks 37 indicate when the body has undertaken a 180° rotation. If required the depth of the body 24 within the incision 11 may be adjusted using horizontal marks 36 as a guide, as previously explained. The distal end 58 of the needle 52 is inserted into the abdomen 16 via passage 28 of the body 24 and with help of the forceps (not shown) and the telescope 22 (see FIG. 1) the first end 82 of thread 38 is mounted on the distal end 58 of the needle 52 (see FIG. 17). The first end 82 of thread 38 is extracted from the abdomen via the passage 28 of the body 24 by extracting the needle 52 from the abdomen and the passage 28 of the body 24 (see FIG. 18). As shown in FIG. 20, subsequently the body 24 is raised to extract the first end 82 of thread 38 from the passage 28.

Referring to FIGS. 21 and 22, in order to close the incision 11 a knot 64 is formed, uniting the first and second ends 82, 84 of the thread 38. Once the body 24 is extracted, first and second ends 82, 84 of the thread 38 are immediately pulled thus closing the incision 11. In this way, gas discharge is avoided and the working and viewing space 20 within the abdomen are maintained. This permits an inspection of the suture line below the abdominal wall using the telescope 22 to ensure no organs have been harmed or sutured onto the inner abdominal surface and/or that the incision 11 has been properly sutured.

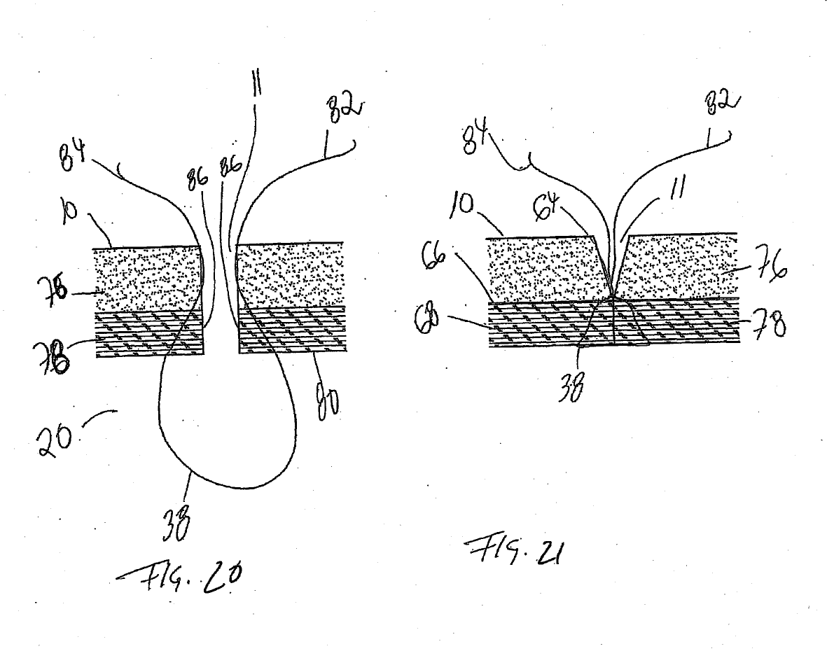

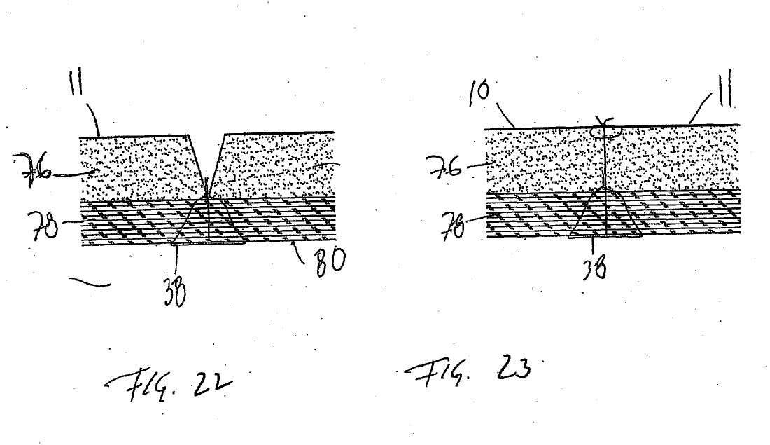

Subsequently, as shown in FIG. 23, the first and second ends 82, 84 of the thread 38 are cut and the upper surface 11 of the abdominal wall 10 is sutured using conventional methods.

Alternatively, if the incision 11 is of a considerable magnitude a plurality of sutures (see FIG. 24) may be required to close the incision 96 (instead of a single suture as previously described). This is accomplished by rotating the body 24 at different angles during each suturing process.

FIG. 24 is a top view of an incision 96 having the body 24 inserted in the incision 96. As shown, three threads 104, 106, 108 have been used to close the incision 96. Each thread 104, 106, 108 comprise first ends 98a, 100a, 102a which have been inserted via a first side of the incision 96 into the abdomen and extracted via a second opposite side of the incision 96 out of the abdomen. Second ends 98b, 100b, 102b of the threads 104, 106, 108 have been located opposite to the first ends 98a, 100a, 102a in accordance to the previously described method.

Referring to FIG. 25, three threads 104, 106, 108 form sutures 110, 112, 114 that are located at spaced intervals along the length of the incision 96. Sutures 110, 112, 114 are formed by rotating the body 24 and thus guiding the needle to the appropriate location on each side of the incision. Suture 110 is formed by guiding the needle to a location 90b close to the first end of the incision in order to insert the first end 98a of the thread 104 inside the abdomen and then rotating the body 24 an angle such that the first end of the thread 98a can be extracted at a location 90a at the opposite side of the incision 96 and counterpart of the location 90b where the first end 98a of the thread 104 was inserted. This procedure can be repeated a plurality of times in order to obtain a plurality of sutures 110, 112, 114 that are located at spaced intervals along the length of the incision 96.

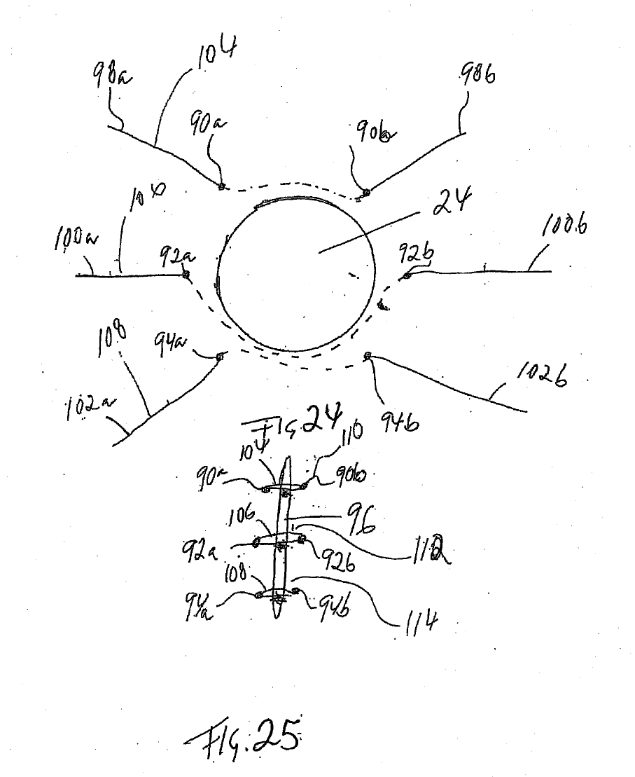

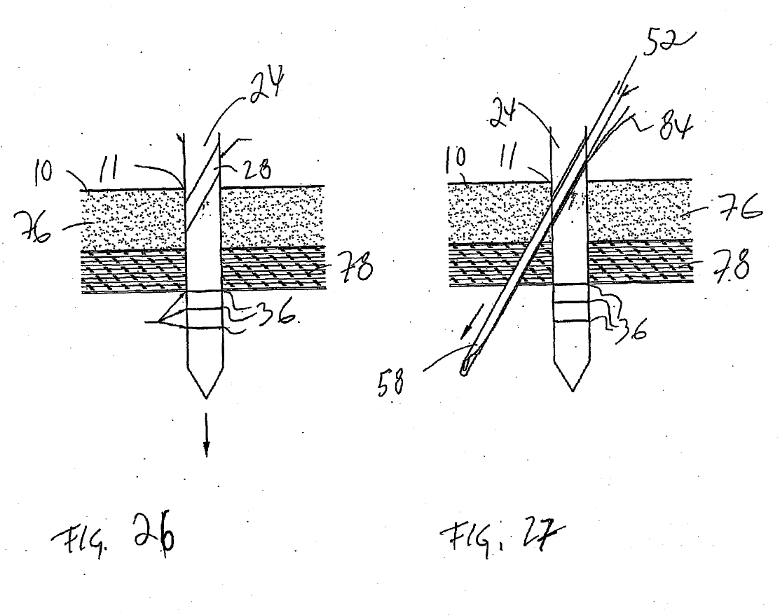

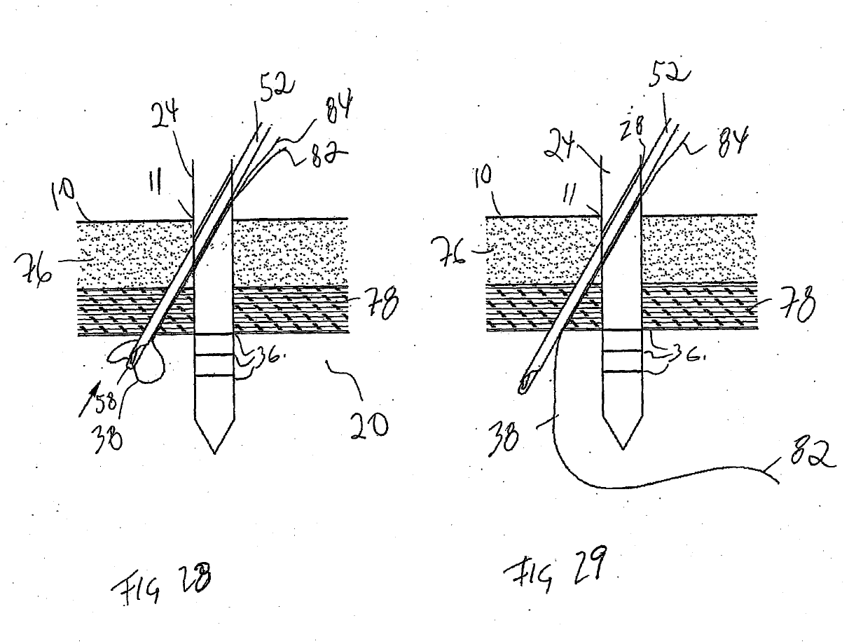

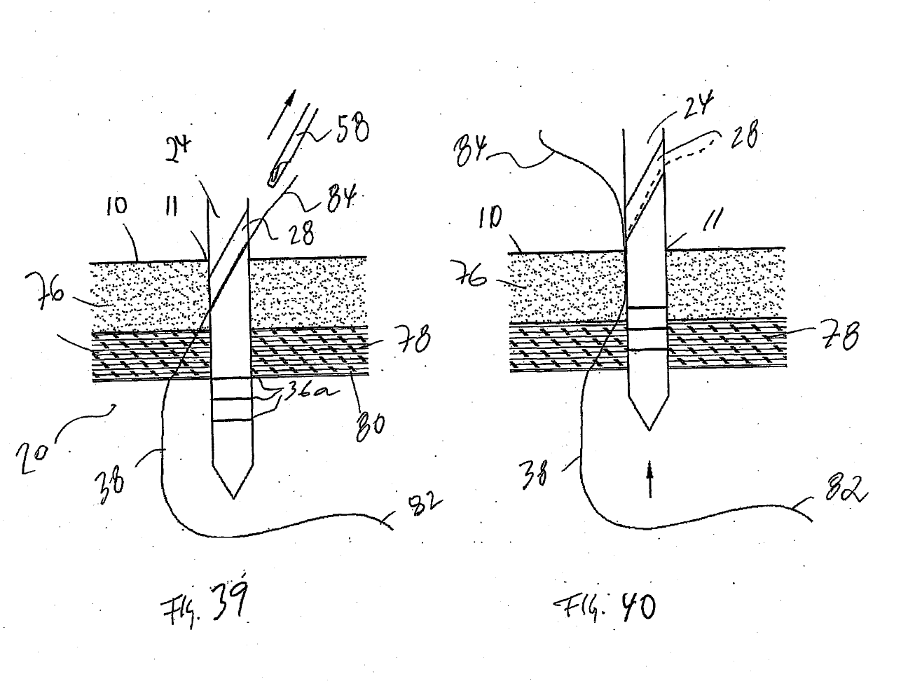

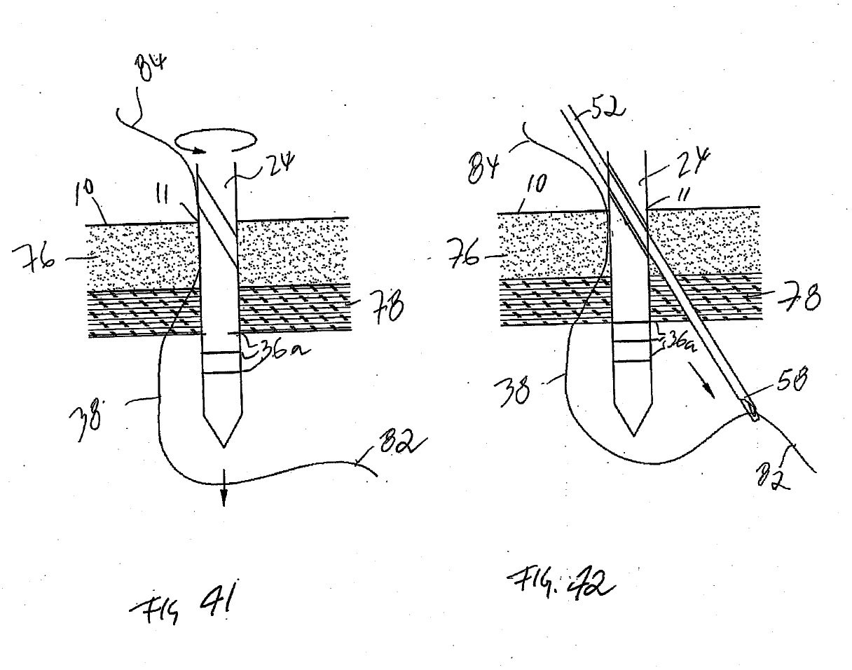

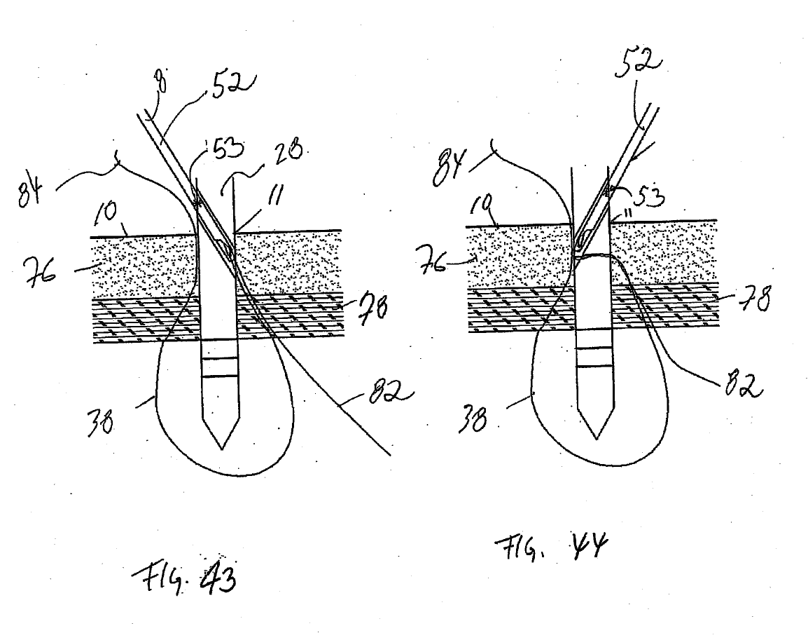

FIGS. 26 to 51 illustrate the method for suturing of an incision using a continuous suture having a single threat. The method shown in FIGS. 26 to 51 is similar to method previously described and shown in FIGS. 12 to 23 and similar reference numerals are used to identify similar parts.

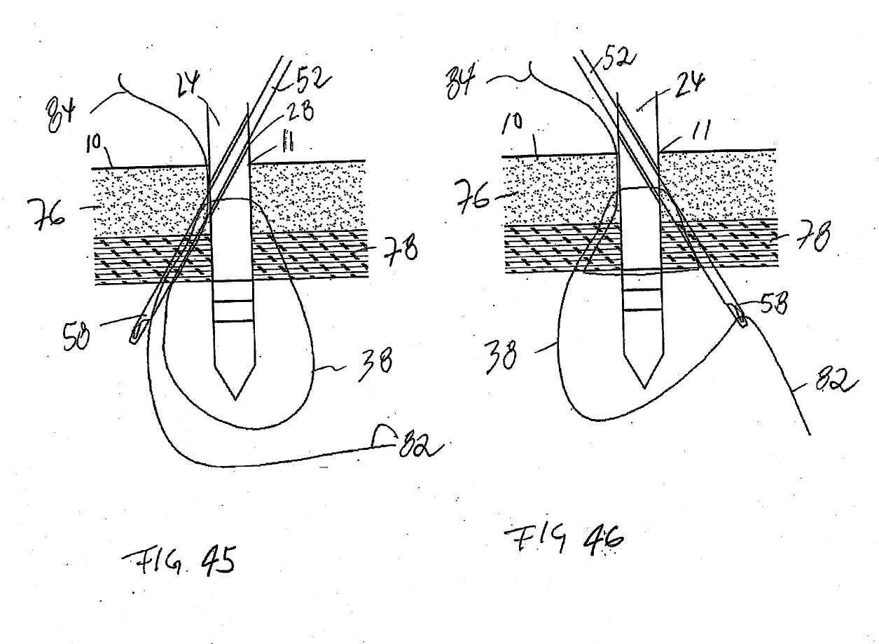

The steps as shown in FIGS. 26 to 42 are substantially identical to the steps shown in FIGS. 12 to 17 with reference to the single suture process. In both methods (single suture process and continuous suture process), after inserting the thread 38 into the abdominal cavity at the first suture site the body 24 is rotated and the needle 52 reinserted at the second suture site (see FIGS. 41 and 42).

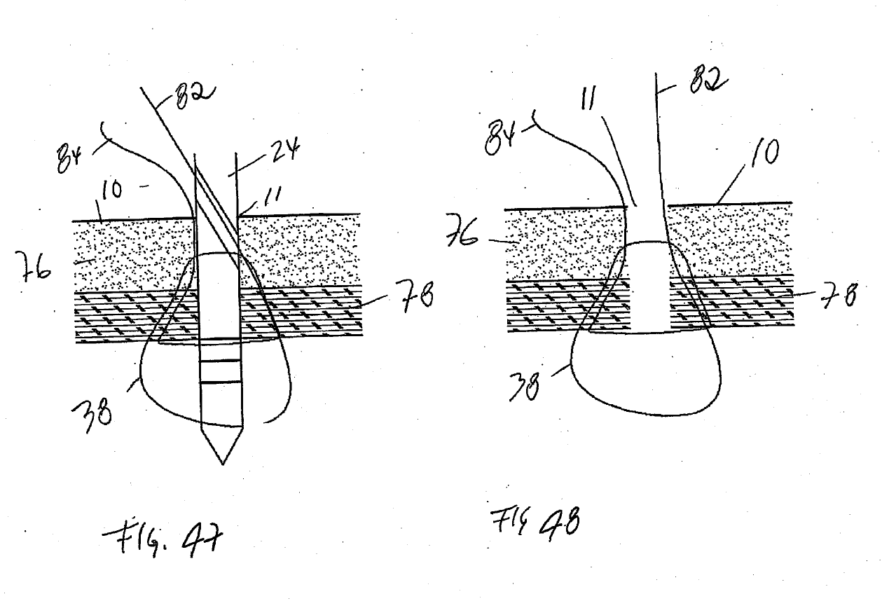

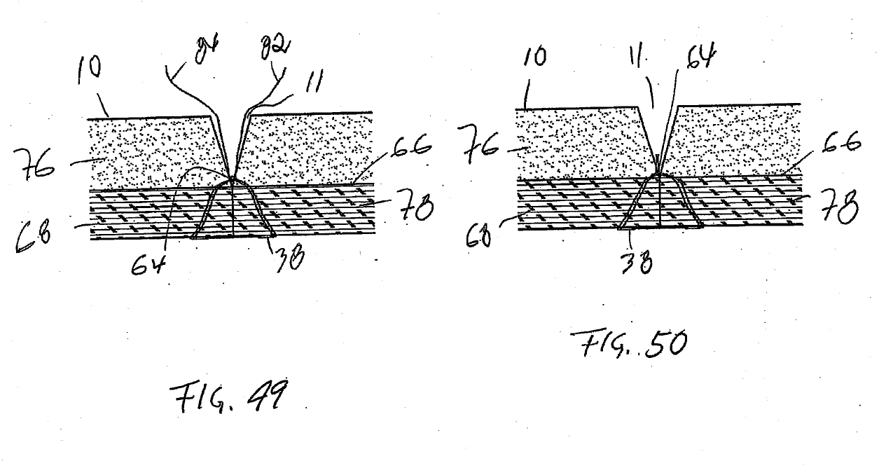

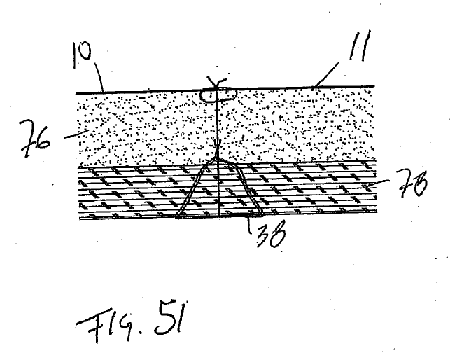

Subsequently, in the continuous suture process, the thread 38 is hooked onto the needle 52 and the needle 52 is not fully withdrawn from the passage 28 but kept within the passage 28 allowing rotation of the body 24 (see FIG. 43). The body 24 can then be rotated again through the desired amount to the third suture site (see FIG. 44). The rotation of the body is monitored from within the abdomen with help of the vertical marks 37. The needle 52 is reinserted to carry the thread 38 back into the abdominal cavity (see FIG. 45). The needle 52 is retrieved to the passage 28 to allow rotation of the body as explained with reference to FIG. 43. Once the body 24 is rotated, the needle 52 may be reinserted into the forth suture site to retrieve the thread 38 (see FIGS. 46 and 47). The body 24 is then extracted from the incision 11 (see FIG. 48) and a knot 64 tied as shown in FIGS. 49 and 50. FIG. 51 shows the sutured incision 11.

Referring to FIG. 43, the needle 52 may comprise a mark 53 which indicates that the distal end of the needle 52 has been fully withdrawn from tissue surrounding the incision 11 but is still located within the passage 28 of the body 24. This arrangement allows rotation of the body 24 (see FIGS. 43 and 44).

This procedure can be repeated a plurality of times in order to obtain a plurality of sutures that are located at spaced intervals along the length of the incision 11 and composed of a single thread 38. In this procedure the continuous sutures can either be arranged to cross from side to side over the incision or pass around the incision back to the starting point thus making a “purse string” type of suture.

Subsequently, as shown in FIGS. 50 and 51, the first and second ends 82, 84 of the thread 38 are cut and the upper surface 11 of the abdominal wall 10 is sutured using conventional methods.

FIGS. 52 to 54 illustrate a method of using the tool 71 shown in FIGS. 10 and 11 to guide a body 24 into an incision 11. An example of a procedure in which the tool 71 might be used is in the repair of an established port site hernia. Initially, a laparoscope is inserted at a location (not shown) away from the port side hernia to be closed. At another location (not shown) a port (for example, a 5 mm port) is inserted in the abdominal wall. The tool 71 is inserted in the port and pushed into the hernia (see FIG. 52). The end 75 of the tool 71 can be felt from the exterior side of the abdominal wall 10. At that location, an incision 11 is made in the abdominal wall 10 in which the surgical instrument, for example, body 24 is inserted (see FIG. 53). The surgical instrument is received by the end 75 of the tool 71. The surgical instrument is then pushed into the abdomen guided by the tool 71. This procedure allows for the hernia sack to be pushed back into the abdomen (see FIG. 54). At this stage the hernia site may be sutured using body 24 and any of the previously described suture procedures.

It is evident that the present invention provides an efficient and effective procedure for suturing incisions. As explained, the inclusion of passage(s) 28 and mark(s) 36, 37 facilitates accurate suturing of incisions. Also, the invention assists that that muscle layers are sutured instead than fat layers. If thick layers of fat are sutured, the tightening of the threat 38 may divide vessels in the fat layer causing bleeding into the patient's tissues. According to the present invention, for example, it is possible to accurately suture 1.5 cm of muscle at the level of the peritoneal lining with minimal suturing of the fat layer.

Moreover, body 24 or, for example, a trocar comprising passages 28, 29 and 31 and marks 36 and 37 may be used to suture port sites having diameters of any size. In very large port sites, the air could be maintained within the abdomen by suturing incision 11 snugly up to the trocar. The ability to suture large ports may be useful for extracting large specimens, such as gallstones or pieces of excised colon, through large ports. Currently, it is necessary to make a separate incision in the abdominal wall only to remove such specimens.

Modifications and variations as would be apparent to a skilled addressee are deemed to be within the scope of the present invention.

Further, it should be appreciated that the scope of the invention is not limited to the scope of the embodiments disclosed. By way of example, the apparatus and method according to the invention may be suitable to suture any type of incision in human or animal bodies.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

1. A surgical device for guiding a needle during suturing of an incision that accesses a body cavity, the surgical device comprising a body for insertion in the incision having a first passage to conduct an end of the needle below a location surrounding the incision, wherein a distal portion of the body is located inside the a body cavity during suturing of the incision and comprises means for indicating the location to which the needle will be conducted.

2. A surgical device according to claim 1 wherein the indicating means may comprise a marking on the distal portion.

3. A surgical device according to claim 1 wherein the location is adjacent to the marking.

4. A surgical device according to claim 1 wherein the first passage traverses obliquely the body.

5. A surgical device according to claim 1 wherein the first passage traverses the body at an angle of about 30° with respect to a side wall of the body.

6. A surgical device according to claim 1 wherein the indicating means comprises a first mark for providing a visual indication of a specific depth of the body within the incision.

7. A surgical device according to claim 1 wherein the indicating means comprises a second mark for providing a visual indication of an angular rotation of the body thereby providing an indication of the location to which the end of the needle will be conducted.

8. A surgical device according to claim 6 wherein the indicating means comprises a series of first marks in spaced apart relation.

9. A surgical device according to claim 7 wherein the indicating means comprises two opposed second marks.

10. A surgical device according to claim 9 wherein each of the opposed second marks extend from the first passage to a distal end of the body.

11. A surgical device according to claim 1 wherein the distal end is configured for entry into the incision.

12. A surgical device according to claim 11 wherein the distal end is of tapered configuration.

13. A surgical device according to claim 1 wherein the body is adapted to sealingly engage the incision.

14. A surgical device according to claim 1 wherein the body further comprising a second passage.

15. A surgical device according to claim 14 wherein the second passage traverses obliquely the body at a second angle relative to the longitudinal axis of the body,

16. A surgical device according to claim 15 wherein the second angle being different than a first angle at which the first passage traverses obliquely the body.

17. A surgical device according to claim 1 wherein the body comprises three or more passages, each of the three or more passages traversing obliquely the body at angles relatively to the longitudinal axis of the body.

18. A surgical device according to claim 17 wherein the angles being different from each other.

19. A surgical device according to claim 14 wherein the passages are arranged to conduct the end of the needle to a same location relative to the incision.

20. A surgical device according to claim 19 wherein the same location is located 1.5 cm radially outwards from the marking

21. A surgical device according to claim 1 wherein the body comprises transparent material.

22. A surgical device according to claim 1 wherein the body is defined by a trocar.

23. A surgical device for guiding a needle during suturing of an incision that accesses a body cavity, the surgical device comprising a body for insertion in the incision having a plurality of passages to conduct an end of the needle below a location surrounding the incision, wherein a distal portion of the body is located inside the body cavity during suturing of the incision comprises means for indicating the location to which the needle will be conducted.

24. A surgical device according to claim 23 wherein the plurality of passage traverses obliquely the body at angles relatively to the longitudinal axis of the body.

25. A surgical device according to claim 24 wherein the angles being different with respect to each other.

26. A dilator for determining the diameter of an incision for use in combination with a surgical device according to any of the preceding claims, wherein the dilator comprises at least one mark indicating the diameter of the dilator at the location of the at least one mark.

27. A tool for guiding the surgical device of claim 1 with respect a biological cavity, the tool comprising at least one end adapted to receive the surgical device.

28. A method for suturing an incision in a biological cavity comprising:
obturating the incision to avoid gas discharge from the biological cavity; and
suturing the incision.

29. A method for suturing an incision in a biological cavity comprising:
inserting a body in the incision having at least one first passage to conduct a first end of a thread to a location below the incision; the body being inserted into the incision until at least one first mark on the body reaches a location of the incision;
inserting a first end of a thread inside the biological cavity through the at least one first passage;
extracting a second end of the thread from the at least one passage;
rotating the body around its longitudinal axis until at least one second mark on the body reaches a location around the incision;
extracting the first end of the thread outside of the biological cavity through the at least one first passage;
extracting the body from the incision;
uniting the first end of the thread with a second end of the thread for closing of the incision.

30. A method according to claim 29 wherein the steps are repeated a plurality of times for finishing to complete closure of the incision using at least one thread.

31. A method according to claim 29 the first end of the thread is inserted into the biological cavity attached to a distal end of a needle.

32. A method according to claim 31 wherein the distal end of the needle comprises fastening means to releasably attach a thread.

33. A method according to claim 32 wherein the fastening means to releasably attach a thread comprise at least one indentation having at least one extension extending into the body of the needle onto which the thread is mounted.

34. A method according to claim 31 wherein, the first end of the thread is removed from the distal end of the needle.

35. A method according to claim 31 the distal end of the needle is extracted from the at least one first passage.

36. A method according to claim 31 distal end of the needle is extracted from incision but kept within the passage of the body so as to allow rotation of the body.

37. A method according to claim 29 further comprising raising the body to extract a second end of the thread from the at least one passage.

38. A method according to claim 37 the body is rotated 180 degrees around its longitudinal axis.

39. A method according to claim 29 wherein the first end of the thread is extracted from the biological cavity through the at least one first passage via the distal end of the needle.

40. A method according to claim 29 further comprising the steps of inserting a telescope into the biological cavity for inspection inside the cavity.

41. A method according to claim 40 wherein the telescope is inserted inside the body for inspection inside the biological cavity.

42. A method according to claim 31, the method further comprises inserting a forceps into the biological cavity for either removing the thread from the needle or hooking the thread to the needle.

43. A method according to claim 42 the first end of the thread is released from the distal end of the needle via the forceps.

44. A method according to claim 42 the first end of the thread is mounted on the distal end of the needle via the forceps.

45. A method according to claim 29 the method further comprises delivering gas into the biological cavity.

46. A method according to claim 29 wherein the method is performed during laparoscopic surgery.

47. A method according to claim 29 wherein the method is performed on a port site incision.

48. A method according to claim 29 wherein the suturing comprises suturing the muscle layer and the peritoneum of the abdomen.

49. A method according to claim 29 the method further comprises the step of selecting the diameter of the body by use of a dilator.

50. A method according to claim 49 the dilator is a graded dilator.

51. A method according to claim 29 the body is defined by a trocar.

52. A method for suturing an incision in a biological cavity comprising
inserting a body in the incision having at least one first passage to conduct a first end of a thread to a location below the incision;
inserting a first end of a thread inside the biological cavity through the at least one first passage;
extracting a second end of the thread from the at least one passage;
rotating the body around its longitudinal axis until at least one second mark on the body reaches a location around the incision;
extracting the first end of the thread outside of the biological cavity through the at least one first passage;
extracting the body from the incision;
uniting the first end of the thread with a second end of the thread for closing of the incision.

53. A method for guiding at least one portion of a surgical instrument into a biological cavity, the method comprising:
inserting a guide having an end adapted to receive at least one end of the surgical instrument;
contacting the end of the guide against a location of a wall of the biological cavity;
making an incision at the location;
inserting the at least one end of the surgical instrument in the incision;
contacting the end of the guide against the at least one end of the surgical instrument; and
inserting the at least one portion of the surgical instrument in the cavity through the incision with assistance of the guide.

54-57. (canceled)
